(19) United States
(12) Patent Application Publication
(10) Pub. No.: US 2021/0299428 A1
(43) Pub. Date: Sep. 30, 2021

(54) UNIVERSAL CAPPING SYSTEM AND THE METHOD OF USE THEREOF

(71) Applicant: George J. Partsch, IV, Fountain Valley, CA (US)

(72) Inventor: George J. Partsch, IV, Fountain Valley, CA (US)

(21) Appl. No.: 17/219,862

(22) Filed: Mar. 31, 2021

Related U.S. Application Data
(60) Provisional application No. 63/003,276, filed on Mar. 31, 2020.

(51) Int. Cl. A61M 39/20; B65D 47/06; A61M 3/02

(52) U.S. Cl. CPC A61M 39/20; A61M 3/0279; B65D 47/06

(57) ABSTRACT

A universal capping system to implement in any bottle-coupling part to sealingly cap over bottle openings of various sizes. Such bottle-coupling part can be a douche nozzle, a liquor pour spout, an olive oil pour spout, and just a bottle closing cap. This universal capping system has a cap top and a downward-extending resilient circular seal.

### CROSS-REFERENCE TO RELATED APPLICATIONS

This application claims priority to, and is a non-provisional patent application of, U.S. application No. 63/003,276, filed on Mar. 31, 2020, now pending, which is hereby incorporated by reference in its entirety.

### FIELD OF THE DISCLOSURE

The present disclosure relates to a universal bottle capping method, more particularly, a universal bottle cap structure that can be implemented on douche nozzle pieces or any bottle-coupling part that is designed to fit on a bottle opening.

### BACKGROUND OF THE DISCLOSURE

There are various types of containers currently available in the market for various uses; they may be made of various materials and its bottle finish may or may not have threads. For example, there are plastic water bottles and soda bottles having threads on their bottle finishes, and there are glass wine bottles without threads on their finishes. Furthermore, the diameter of the bottle rim opening can vary. The thread on the bottles can each have a different pitch, different thread height, and different thread angles. Generally, each bottle must have a cap that matches the properties of the bottle finish in order for the cap to work properly.

There remains a need to provide a universally fitting cap that sealingly fit over a variety of bottle finishes. There also remains a need to implement such universal capping structure on any bottle-coupling part (e.g., douche nozzle tip) so that the bottle-coupling part can fit over a variety of bottle finishes.

All referenced patents, applications and literatures are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The herein disclosed embodiments may seek to satisfy one or more of the above-mentioned needs. Although the present embodiments may obviate one or more of the above-mentioned needs, it should be understood that some aspects of the embodiments might not necessarily obviate them.

### BRIEF SUMMARY OF THE DISCLOSURE

In a general implementation, this disclosure is regarding a universal capping system that can be implemented on any bottle-coupling part which is generally meant to couple to an opening of a bottle. For example, this bottle-coupling part can be a detachable douche nozzle, a liquor pour spout, an olive oil spout. This capping system can also be implemented simply as a cap alone.

In another aspect combinable with the general implementation, the capping system can have a cap top, a downward-extending seal disposed on a bottom side of the cap top.

In another aspect combinable with the general implementation, the downward-extending seal can be circular in shape.

In another aspect combinable with the general implementation, the circular seal can have cut-outs to act as pressure/tension reliefs.

In another aspect combinable with the general implementation, the circular seal can have a resilient property and can bend inwardly when it fits onto a bottle finish that has an inner diameter smaller than the outer diameter of the circular seal.

In another aspect combinable with the general implementation, there can be a circular outside wall attached to the cap top and extends downward. In some embodiments, this circular outside wall has inner threads. This inner thread can matingly fit over the thread on a bottle finish. In other embodiments, with the inner thread in place, the cap can still sealingly fit over a bottle finish without any thread or with a thread of non-matching pitch and/or angle. In still other embodiments, the circular outside wall has no inner threads and the cap may still sealingly fit over a bottle finish, as will be described in more details later.

In another aspect combinable with the general implementation, the circular outside wall can have at least one vertical open slit, or cut out, to function as a pressure/tension relief.

In another aspect combinable with the general implementation, the circular outside wall can have a resilient property and can bend outwardly when the cap is fitted over a bottle finish that has an outer diameter larger than an inner diameter of the outside wall.

In another aspect combinable with the general implementation, the circular outside wall can be relatively shorter. In another embodiment, the circular outside wall can be relatively longer. In other words, circular outside walls of many lengths are explicitly contemplated, all of which can universally and sealingly cap over a bottle finish. In one example, the outside wall can have a length that extends beyond the lowest part of the circular seal. In another example, the outside wall can have a length that does not extend beyond the lowest part of the circular seal. In still another example, there is no circular outside wall at all.

Similar to the outside wall, the circular seal can be relatively shorter. In another embodiment, the circular seal can be relatively longer. In other words, circular seals of many lengths are explicitly contemplated, all of which can function for the cap to universally and sealingly cap over a bottle finish. In one example, the seal can have a length that extends beyond the lowest part of the circular outside wall. In another example, the seal can have a length that does not extend beyond the lowest part of the circular outside wall. In still another example, no seal necessary to sealingly and universally cap over a cap finish.

In another aspect combinable with the general implementation, the seal can have a hook tip disposed near its lower rim.

In another aspect combinable with the general implementation, the circular seal does not have to form a complete circle and can be separate arcuate pieces forming a near complete circle.

In another aspect combinable with the general implementation, the circular seal can have a bottom taper portion that tapers inward.

In another aspect combinable with the general implementation, the circular seal can have an increasingly smaller diameter from top to bottom.

In another aspect combinable with the general implementation, the circular seal has a vertical height/length sufficiently long to reach beyond the lowest point of a bottle neck when fully fitted onto the bottle finish. In one example, the seal has a hook shape capable of locking with the lowest point the bottle neck.

In another aspect combinable with the general implementation, the force of pushing the cap down creates a seal in at least one of the following locations: 1) between the rim of the bottle opening and the outside wall of the seal, 2) between the rim of the bottle opening and the inside wall of the outside wall, 3) between the rim of the bottle and the cap roof that converges with the outside wall, 4) between the thread of the bottle and the thread of the outside wall, and 5) between a vertical surface of the seal and the inside surface of the bottle finish.

While this specification may contain many specific implementation details (e.g., douche nozzle, liquor pour spout, olive oil pour spout), these should not be construed as limitations on the scope of any inventions or of what may be claimed, but rather as descriptions of features specific to particular implementations of particular inventions.

Some features may be described above and below as acting in certain combinations and even initially claimed as such, one or more features from a claimed combination can in some cases be excised from the combination, and the claimed combination may be directed to a subcombination or variation of a subcombination.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

It should be noted that the drawing figures may be in simplified form and might not be to precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1 is a side cross-sectional view of an embodiment of the contemplated universal capping system having a circular outside wall with a thread and a shorter circular seal having a hook tip, according to an aspect of the embodiment.

FIG. 2 is a side cross-sectional view of another embodiment of the contemplated universal capping system having a circular outside wall with a thread and a slightly longer circular seal having a curved profile and pressure/tension relief cut outs, according to an aspect of the embodiment.

FIG. 3 is a side cross-sectional view of yet another embodiment of the contemplated universal capping system having a circular outside wall with a thread and pressure/tension relief cuts out, and a slightly longer circular seal having a curved profile and pressure/tension relief cut outs, according to an aspect of the embodiment.

FIG. 4 is a side cross-sectional view of still another embodiment of the contemplated universal capping system having a circular outside wall with a thread, and a much longer circular seal having a curved profile, a taper, and pressure/tension relief cut outs, according to an aspect of the embodiment.

FIG. 5 is a side cross-sectional view of still another embodiment of the contemplated universal capping system having a circular outside wall with a thread and pressure/tension relief cut outs, and a much longer circular seal having a curved profile, a taper, and pressure/tension relief cut outs, wherein the capping system is sealingly fitted over a bottle finish that is slightly larger, according to an aspect of the embodiment.

FIG. 6 is a side cross-sectional view of another embodiment of the contemplated universal capping system having a circular outside wall with a thread, and a much longer circular seal having a curved profile, a hook end, and pressure/tension relief cut outs, according to an aspect of the embodiment.

FIG. 7 is a side cross-sectional view of the embodiment shown in FIG. 6 fitted over the bottle finish that is slightly smaller, thereby causing the seal to distort and effectively seal over the bottle rim, according to an aspect of the embodiment.

FIG. 8 is a side cross-sectional view of still another embodiment of the contemplated universal capping system having a circular outside wall with a thread, and a much longer circular seal having a hook end, wherein the hook end is sufficiently long to reach the end of the bottle neck and engages with it to effectively lock and seal, according to an aspect of the embodiment.

FIG. 9 is a side cross-sectional view of yet another embodiment of the contemplated universal capping system having a circular outside wall with a thread, and a much longer circular seal having a tapered end and a decreasing diameter that facilitates insertion into a smaller bottle rim, according to an aspect of the embodiment.

FIG. 10 is a bottom perspective view of still another embodiment of the contemplated universal capping system having a circular outside wall having a thread, a circular seal having two pressure/tension reliefs, and a liquid dispensing opening, according to an aspect of the embodiment.

FIG. 11 is a side bottom perspective view of the embodiment of FIG. 10, showing a liquid entry port, according to an aspect of the embodiment.

FIG. 12 is side cross-sectional view of still another embodiment of the contemplated universal capping system having no circular outside wall at all. This embodiment has a circular seal having at least one pressure/tension relief cut out. The circular seal alone can be sufficient to sealingly cap over a bottle finish, according to an aspect of the embodiment.

FIG. 13 is a perspective view of elongated douche nozzles implementing the contemplated universal capping system, according to an aspect of the embodiment.

FIG. 14 illustrates prior art bottles.

FIG. 15 illustrates a cross-sectional view of a prior art bottle finish.

The following call-out list of elements in the drawing can be a useful guide when referencing the elements of the drawing figures:
1 Cap
2 Cap Top
3 Nozzle
4 Outside wall
5 Cap Rim
6 Cap Thread
7 Converging Roof
8 Liquid Dispensing Opening
9 Liquid Exit Port
10 Seal
11 Seal Rim
13 Seal Hook Tip
22A Seal Relief
22B Top of Seal Relief
24A Outside Wall Relief
24B Top of Outside Wall Relief
31 Bottle Rim
32 Finish
33 Finish Thread
34 Neck
35 Neck Ring
36 Shoulder
37 Body
38 Bottom
A A Dimension
B B Dimension
E E Dimension
I I Dimension
T T Dimension

### DETAILED DESCRIPTION OF THE EMBODIMENTS

The different aspects of the various embodiments can now be better understood by turning to the following detailed description. These embodiments are presented as illustrated examples of the inventive subject matter as defined in the claims. It is expressly understood that the inventive subject matter as defined by the claims may be broader than the illustrated embodiments described below. It will, nevertheless, be understood that no limitation of the scope of this disclosure is thereby intended; any alterations and further modifications of the described or illustrated embodiments and any further applications of the principles of the disclosure as illustrated therein are contemplated as would normally occur to one skilled in the art to which the disclosure relates.

The inventor has discovered a capping system that can universally fit onto bottle finishes of various types and sizes. The capping system is not limited to merely a cap to seal a bottle. The contemplated capping system can be part of any bottle-coupling part such as a douche nozzle head or an enema applicator to fit over a typical water bottle. The word “nozzle” used herein refers to an opening through which liquid can be dispensed. It can have various shapes and sizes. It may or may not include a cylindrical spout. It can be merely an opening on an otherwise smooth surface. Furthermore, the contemplated capping system can be part of any other conceivable bottle-coupling part to fit over a bottle, such as a baby bottle sippy, a nipple for baby bottles, a liquor pour spout, an olive oil spout, or a connector to sealingly connect an item to a bottle finish.

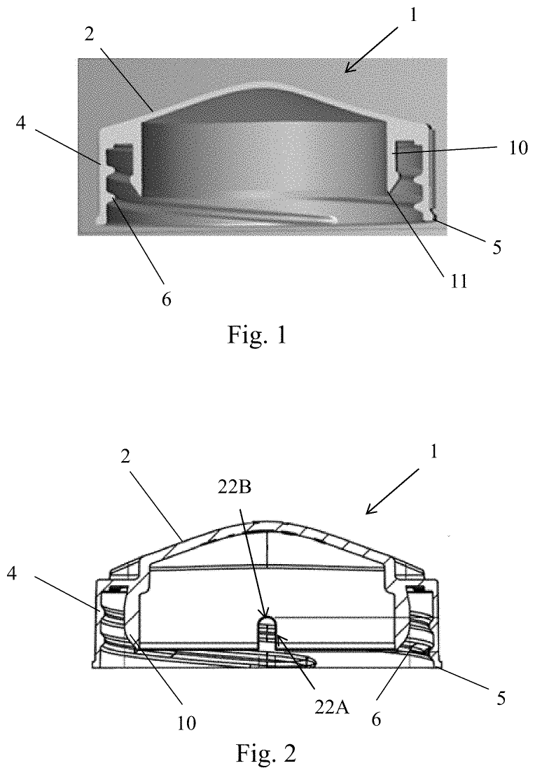

Referring first to FIG. 1, an implementation of the universal capping system 1 is shown. Here, the universal capping system can have a cap top 2 which is a roof that can be generally tapered. The cap top 2 has a round shape from a top view (not shown). The circumference of the cap top 2 is coupled to a circular outside wall 4. The circular outside wall 4 is similar to a typically outside wall of generally know water bottle caps. The outside wall 4 can be attached to the cap top 2 and can extends downward generally in a direction parallel to the longitudinal axis of the bottle. The circular outside wall 4 can have a bottommost edge forming a cap rim 5.

On the side of the outside wall facing the exterior environment there can be striations (not shown) or grip lines to create friction with a user's fingers. On the opposite side (the side facing the interior), there can be a cap thread 6.

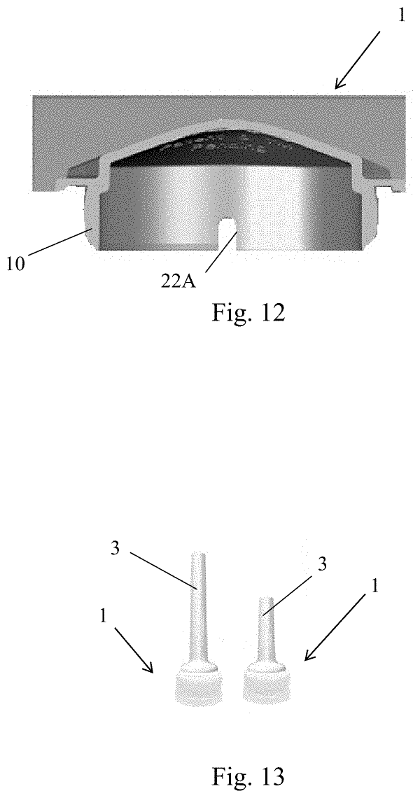

As will be described later (see FIG. 12), there can be embodiments where cap thread 6 may not be necessary thus not present. Also, there can be embodiments where the outside wall 4 can be entirely missing or is much shorter than what is shown in FIG. 1. The bottommost part of the outside wall 4 forms a cap rim 5. From a bottom view, the cap rim 5 is circular.

The circular, or cylindrical outside wall 4 can have a resilient property and can bend outwardly when fitted over a bottle finish 32 having a T or E dimension (see FIG. 15) that is larger than the inner diameter of the outside wall 4.

Returning now to FIG. 1, there can be a circular or cylindrical seal 10 coupled to the cap top 2 and extends downwardly. It should be noted that throughout this disclosure the seal 10 and the outside wall 4 are sometimes described as being circular, and sometimes described as being cylindrical. They are circular in that from a bottom view, the seal 10 and the outside wall 4 can each have a circular shape. They are cylindrical in that from a perspective view, the seal 10 and the outside wall 4 can have a cylindrical structure.

In most embodiments, the seal 10 extends parallelly to a longitudinal axis of the bottle. In some embodiments as will be described later, the seal 10 can have a taper or an inward tilt (see FIG. 9).

The circular seal 10 can have a resilient property and can bend inwardly when needed. For example, when the capping system 1 is fitted onto a bottle finish 32 that has an inner diameter (I dimension, see FIG. 15) that is smaller than the outer diameter of the circular seal 10.

Although the seal 10 can have a resilient property, the seal 10 can also be made of material with a harder durometer reading. Such harder material may provide less surface friction but may endure more forceful inadvertent manipulation. In this way, less material can be used for the seal 10 yet the seal can still provide sufficient and sturdy connection to the bottle finish 32.

The seal 10 has a circular shape from a bottom view of the capping system 1. The circular seal 10 has an inner diameter and an outer diameter. The seal 10 and the outside wall 4 share the same center point. The seal 10, however, has a smaller diameter than the outside wall 2. The circular seal 10 and the outside wall 2 are concentrically arranged such that from a bottom view (not shown), the seal 10 would appear to nestle within the outside wall 2.

Here, the seal 10 is disposed and extends from the underside of the cap top 2. There are many contemplated lengths and shapes of the seal 10, as will be described in more details later.

The seal 10 can have various thicknesses. In one embodiment, the seal 10 has a uniformed thickness of between 0.10 mm and 10.00 mm; in another embodiment, a uniformed thickness of between 0.50 mm and 8.00 mm; in still another embodiment, a uniformed thickness of between 0.50 mm and 5.00 mm; in yet another embodiment, a uniformed thickness of between 0.30 mm and 2.00 mm; in still yet another embodiment, a uniformed thickness of between 0.5 mm-3.0 mm.

The seal 10 can create lateral pressure on the inside of the bottle neck. The lateral pressure created can minimize the chances that the capping system would pop off during use. It may be sufficiently tight so that it would be single use only.

In one embodiment, there can be a thicker seal 10 which may eliminate the need for a perfect fit between the outside wall 4 and a bottle finish 32. Some uses of the capping system 1 may require a thicker seal 10 so as to create more lateral force to keep the capping system 1 in place when the user squeezes the bottle to squirt out the liquid. In one example, an enema applicator having a long nozzle and is the contemplated universal capping system 1 can have its nozzle safely inserted into the rectum without accidentally popping off the capping system 1 from the bottle finish 32. A longer and thicker seal 10 in this particular case can help keep the nozzle in an upright position relatively to the bottle even when the patient's body may be inadvertently pressing against the nozzle side ways.

In the embodiment of FIG. 1, the seal 10 has a seal rim 11. The seal rim 11 is the bottommost edge of the seal 10. The seal rim 11 in the embodiment of FIG. 1 cannot extend beyond the lowest point of the outside wall 2. In other words, when the universal capping system 1 is placed right-side up on a flat surface, the outside wall 4 would appear longer than the seal 10.

The seal rim 11 as shown in FIG. 1 can be pointy, formed by a tapered end. The tapered end tapers inwards. The tapered end can have a cross-sectional shape in the shape of a half-arrowhead, forming a hook, as will be described later. The tapered end from a respective view, can form a frusto-conical shape.

In the embodiment shown in FIG. 1, both the outside wall 4 and the seal 10 are contemplated to have a continuous circular configuration. That is, they do not have cutoffs or relieves. It should be understood that in some embodiments, the outside wall 4 and the seal 10 can be comprised of separate arcuate pieces forming a discontinuing circle (not shown). For example, the seal 10 can be comprised of 2-3 separate arcuate shaped pieces.

In the embodiment of FIG. 2, the seal 10 is a single continuous circular/cylindrical piece. The seal 10 here has a cross-sectional shape that has a rounded-off outer surface with a mid-portion that slightly bulges out. The bulge can create increased frictional fit with the interior of a bottle finish 32. When the capping system 1 here is fitted over a bottle finish 32 with a smaller I dimension (FIG. 15), the seal 10 may bend inwards to accommodate the smaller I dimension. As the seal 10 bends inward, the bulged mid-portion continues to make frictional contact with the interior of the bottle finish 32, effectively sealing the bottle.

Many of the embodiments described herein allows a fairly tight frictional contact between the seal 10 or the outside wall and the bottle finish 32. Some of these frictional contacts can be sufficiently tight and it can be thrown away along with the bottle after a single use.

In the embodiment of FIG. 2, there can be one or more pressure/tension relieves 22A. In other embodiments, there can be no pressure/tension 22A relieves at all. A pressure/tension relief 22A of the seal 10 can be a cut out, a notch, a slit, or a physical separation in the seal rim 11. The pressure/tension relief 22A as shown in FIG. 2 causes the otherwise continuous circular seal rim 11 to be separated into arcuate pieces of seal rim 11. The seal 10 itself, however, remains a single circular piece. One contemplated pressure/tension relief 22A can be a cove having an upper end 22B. This cove can be of various length and widths. In FIG. 2, the upper end 22B of the cove is at a vertical height that does not go beyond the base of the seal 10, and the base of the seal 10 is defined as where the seal 10 converges with the top cap 2. In another embodiment, the pressure/tension relief 22A has a length of about half of the vertical length of the seal 10 (from the base of the seal 10 to the seal rim 11).

The seal relief 22A can have a wider width at the top and a narrower width towards the bottom (e.g., FIG. 9). In another embodiment, it can have a uniformed width of between 0.1 mm and 3 mm; in another embodiment, a uniformed width of between 0.1 mm and 2 mm; in still yet another embodiment, a uniformed width of between 0.3 mm and 1 mm. The relief 22A can have a length between 2 mm and 15 mm; in another embodiment, a length between 4 mm and 10 mm; in still another embodiment, a length between 5 mm and 8 mm.

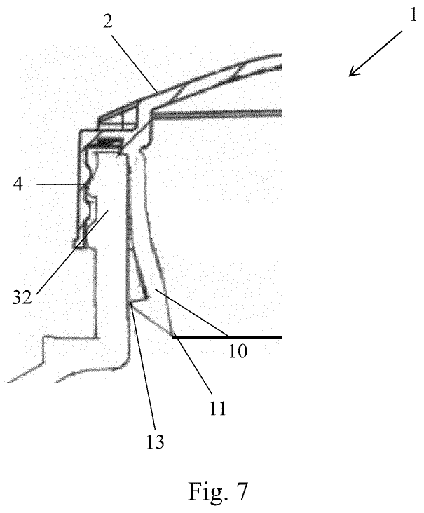

A pressure/tension relief 22A such as that shown in FIG. 2 can improve the resiliency of the seal 10, allowing the seal rim 11 to radially move inward while minimizing unwanted contortion in the shape of the seal 10 or seal rim 11. In one example as shown in FIG. 7, when the capping system 1 is fitted over a slightly larger finish 32, the seal rim 11 will need to move inward to allow the initial insertion of the finish 32. Without having pressure/tension relieves 22A, the seal rim 11 may still bend inward, but would contort unevenly into a non-circular shape. Such contortion can decrease the ability of the seal 10 to prevent a leak.

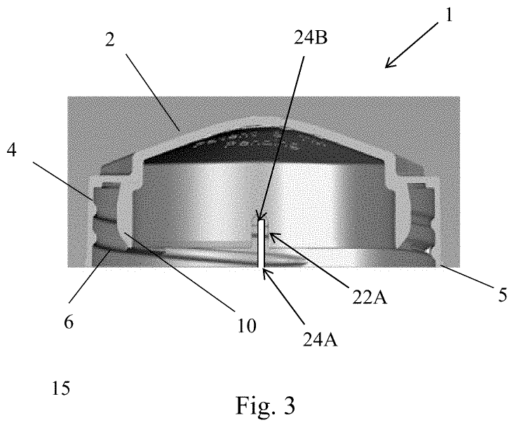

Alternatively or additionally, there can be at least one pressure/tension relief 24A disposed on the outside wall 4 as shown in FIG. 3. The function of this pressure/tension relief 24A is similar to the pressure/tension relief 22A as described above.

A pressure/tension relief 24A of the outside wall 4 can be a cut out, a notch, a slit, or a physical separation in the outside wall 4. The pressure/tension relief 24A as shown in FIG. 3 causes the otherwise continuous cap 5 to be separated into arcuate pieces of cap rim 5. The outside wall 4 itself, however, remains a single cylindrical piece. One contemplated pressure/tension relief 24A can be a cove having an upper end 24B. This cove can be of various length and widths. In FIG. 3, the upper end 24B of the cove is at a vertical height that does not go beyond the base of the seal 10, and the base of the seal 10 is defined as where the seal 10 converges with the top cap 2.

In another embodiment, the pressure/tension relief 24A of the outside wall 4 is no higher than the base of the outside wall 4. In another embodiment, the pressure/tension relief 24A of the outside wall 4 is about half the length of the outside wall 4. In yet another embodiment, the pressure/tension relief 24A of the outside wall 4 is shorter than half the length of the outside wall 4. The base of the outside wall 4 is defined as where the outside wall 4 converges with the cap top 2.

The outside wall 4 can have a uniformed thickness (not including the thickness/height of any thread) of between 0.30 mm and 10.00 mm; in another embodiment, a uniformed thickness of between 0.50 mm and 8.00 mm; in still another embodiment, a uniformed thickness of between 0.30 mm and 5.00 mm; in yet another embodiment, a uniformed thickness of between 0.70 mm and 2.00 mm. Alternatively, the outside wall 4 can have varied thickness throughout the wall 4 in a particular embodiment.

In other embodiments, the exterior of the outside wall 4 has no grip lines. This is to ensure the capping system is less invasive for uses that may require most or part of the capping system to enter a body cavity. For example, the vagina or anus of a patient. The grip lines are also not necessary in some embodiments if they are meant for single use applications.

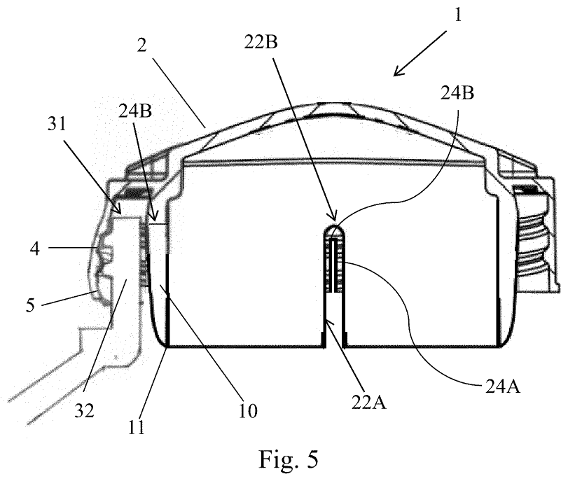

FIG. 5 illustrates one example of the pressure/tension relief 24A in operation. In FIG. 5, the capping system 1 is fitted over a slightly larger finish 32 thereby causing the outside wall 4 to expand radially. The pressure/tension relief 24A allows the cap rim 5 to expand into a slightly larger diameter and allows the finish to 32 to make a sealing contact with the interior of the outside wall 4. In some embodiments, the sealing contact is made between the interior of the finish 32 and the outside of the seal 10.

The seal 10 may have various cross-sectional shapes. In one embodiment, the cross-sectional shape of the seal 10 can be a half-arrowhead (i.e., FIG. 1). In another embodiment, the cross-sectional shape of the seal 10 can be flat on the interior side and arcuate on the exterior side (i.e., FIG. 3).

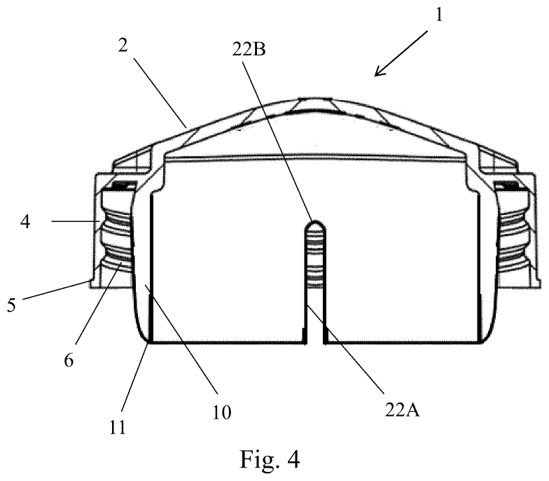

The seal 10 is contemplated to have various lengths. In some embodiments, the seal rim 11 does not reach beyond the cap rim (e.g., FIGS. 1-3). In other embodiments, the seal rim 11 has about the same reach as the cap rim (not shown). In still yet other embodiments, the seal rim 11 can reach farther than the cap rim 5 (e.g., FIGS. 4-9).

Returning now to FIG. 5, if the capping system 1 of FIG. 5 is instead fitted over a slightly smaller bottle finish 32, the long seal 10 may make the coupling easier. This is because the over-extended portion of the seal rim 11 may have a diameter that is about the same or slightly larger than the diameter of the bottle opening (I, I dimension of FIG. 15). By having the seal rim 11 over-extended and visible, a user may more easily maneuver and insert the seal rim 11 into the bottle opening.

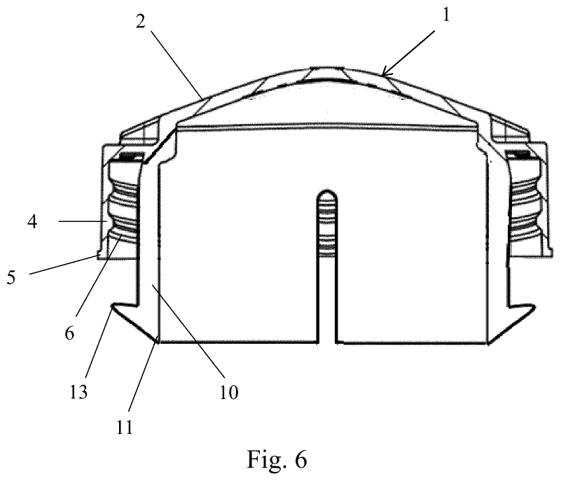

As for FIG. 6, this embodiment provides an over-extended seal rim 11 and it has a cross-sectional shape of a half-arrow. The half-arrow has a hook tip 13 on the exterior side of the seal 10. The hook tip 13 creates a rim surrounding the lower portion of the seal. This rim created by the hook tip 13 can have a diameter at rest (as shown in FIG. 6) that is larger than the diameter of the cylindrical outer surface of the seal 10. When this embodiment is fitted over a slightly smaller bottle finish 32 as shown in FIG. 7, the seal 10 can bend inward to accommodate the slightly smaller bottle opening (I, I dimension of FIG. 15). The hook point 13 can create a frictional seal with the interior wall of the bottle finish 32. Here in FIG. 7, there may also be a frictional seal created between the exterior of the bottle finish 32 and the interior of the outside wall 4.

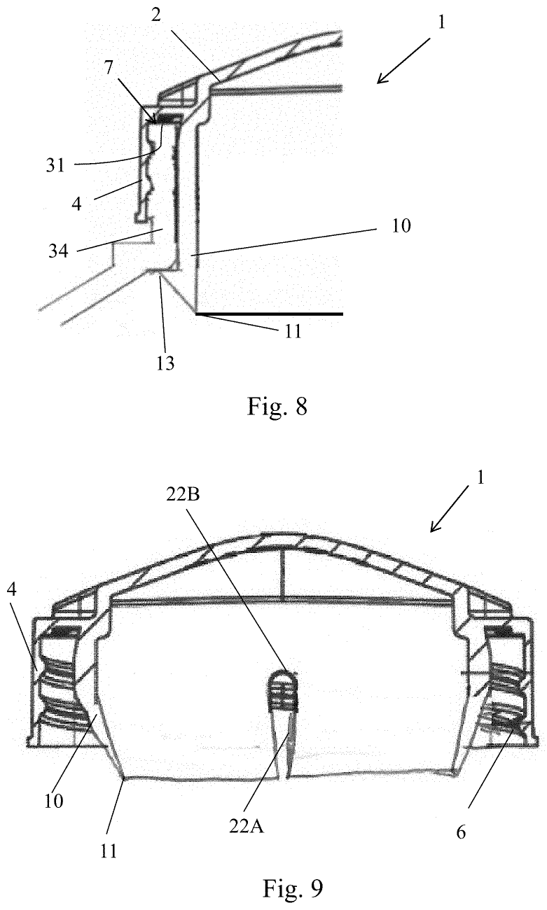

The contemplated over-extended seal rim 13 can also be sufficiently long such that the hook point 13 fits over the bottom terminal end of the bottle finish 32 or bottle neck 34 to “click” into place and lock the capping system 1 onto the bottom (see FIG. 8). In FIG. 8, by locking into place, more frictional locking surfaces are created, further improving the capping system's sealing ability.

Referring now to FIG. 9, a contemplated seal 10 can have an initial inward bend at rest such that the seal rim 11 has a smaller diameter than the diameter of the seal 10 at the base of the seal 10. In other words, the interior surface of this particular seal 10 is not a cylindrical shape but a cone shape. The circumference of this particular seal 10 decreases towards the seal rim 11. This inward bend can facilitate the insertion into a bottle finish 32.

In FIG. 9, the seal 10 may have a relative thickness and variation in thickness that is as shown. In another embodiment, it may be substantially thicker than shown towards the top half of the seal 10 so that the thicker portion of the seal 10 can provide a strong lateral force to the interior of the bottle finish 32. In other words, the top portion of the seal 10 may require little flexing, when compared to the seal rim 11 portion. Being less flexible, the top portion of the seal 10 can sufficiently make frictional engagement with a bottle finish 32. In one way, the thicker portion of the seal 10 can work like a cork stop. The more flexible seal rim 11, on the other hand, is more flexible so that a user may easily insert it into the bottle finish 32.

Also contemplated is for the embodiment shown in FIG. 9 to remain having a tapered lower portion and a decreasing diameter, and additionally having a uniformed thickness throughout the entire seal 10.

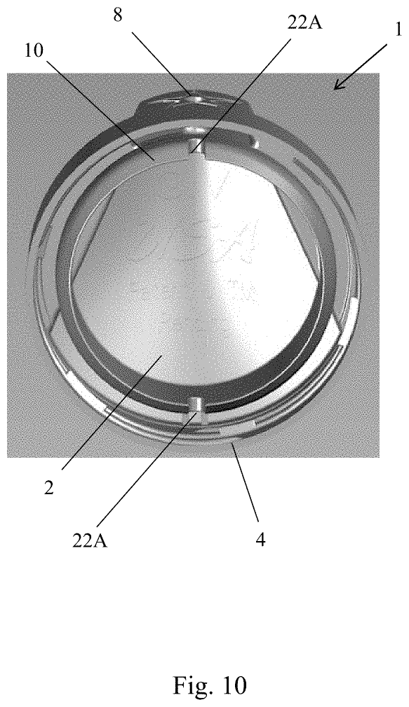

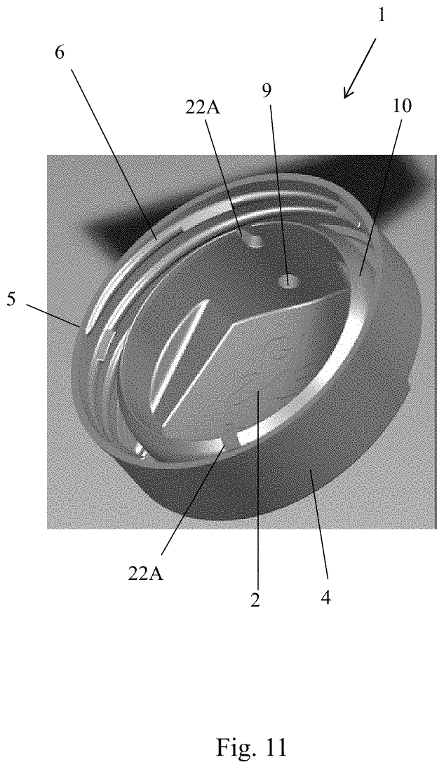

Referring now to FIGS. 10-11, as mentioned earlier, the capping system 1 may be implemented on various types of parts designed to couple to a bottle. These bottom views of one embodiment show a capping system 1 having a seal 10 with two opposing seal relieves 22A, and an outside wall 4 that has no pressure/tension relieves. On the interior side of the capping system there can be a liquid exit port 9 which leads to the liquid dispensing opening 8 on the outside of the capping system 1. In one embodiment, this opening 8 can be a slot, a line opening that is about 0.030 inches tall and 3 mm wide. This particular configuration describes a portable douche cap which can be universally fitted over a water bottle of various bottle finish sizes. FIG. 13 shows the universal capping system 1 implemented on two portable douche caps each having a nozzle 3 where one is longer than the other.

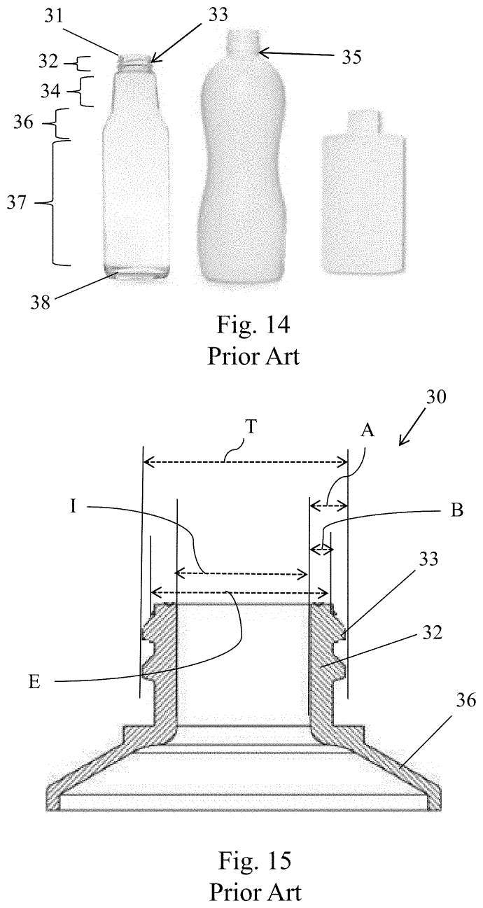

FIG. 14 illustrates typical bottles of various bottle finish sizes. A bottle, whether it is made of glass, plastic, or other materials, typically has a bottle rim 31, a bottle finish 32, a finish thread 33 on the bottle finish 32, a bottle neck 34, a bottle shoulder 36, a bottle body 37, and a bottle bottom 38. Some bottles may have a neck ring 35. Referring now to FIG. 15, a bottle finish typically has the following dimensions: T dimension, a diameter measured between the outer most part of the finish thread 33 on opposite ends. I dimension, a diameter measured between the opposing interior walls of the bottle finish 32. E dimension, a diameter measured between the bases of the finish thread 33 on opposite sides. The difference between the E and T dimensions divided by two determines the thread depth. “A” dimension, a thickness measured between the interior wall of the finish 32 and the outer most point of the finish thread 33. B dimension, which is a thickness of the finish 32 without the finish thread 33. The finish thread 33 can have a different angle pitch from one bottle to another. No matter the differences in these dimensions and angles, any one of the many contemplated universal capping systems 1 can accommodate and make a sealing coupling to these bottles.

Many alterations and modifications may be made by those having ordinary skill in the art without departing from the spirit and scope of the disclosed embodiments. Therefore, it must be understood that the illustrated embodiments have been set forth only for the purposes of example and that it should not be taken as limiting the embodiments as defined by the following claims. For example, notwithstanding the fact that the elements of a claim are set forth below in a certain combination, it must be expressly understood that the embodiment includes other combinations of fewer, more or different elements, which are disclosed herein even when not initially claimed in such combinations.

Thus, specific embodiments and applications of a universal capping system have been disclosed. The disclosed embodiments is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the embodiments. In addition, where the specification and claims refer to at least one of something selected from the group consisting of A, B, C . . . and N, the text should be interpreted as requiring at least one element from the group which includes N, not A plus N, or B plus N, etc.

What is claimed is:

1. A universal capping system to implement in a bottle-coupling part to sealingly cap over a variety of bottle opening sizes, said capping system comprising:
a cap having a cap top;
a downward-extending circular seal disposed on a bottom side of the cap top;
the circular seal has an inner diameter and an outer diameter;
wherein the circular seal is resilient and can bend inwardly when the cap is fitted onto a bottle finish that has an inner diameter that is smaller than the outer diameter of the circular seal;
wherein the circular seal has a bottommost edge forming a seal rim; and
wherein the bottle-coupling part has an outside surface having a liquid-dispensing opening, and an interior surface having a liquid-exiting port.

2. The universal capping system as recited in claim 1 further comprising a circular outside wall attached to the cap top and extends downward, and wherein the circular outside wall has a bottommost edge forming a cap rim.

3. The universal capping system as recited in claim 2, wherein the circular outside wall has at least one vertical open slit forming an outside wall relief.

4. The universal capping system as recited in claim 2, wherein the circular outside wall is resilient and can bend outwardly when fitted over a bend inwardly when the cap is fitted over a bottle finish that has an outer diameter that is larger than an inner diameter of the outside wall.

5. The universal capping system as recited in claim 2, wherein the circular outside wall has a cap thread disposed on an inside surface of the circular outside wall.

6. The universal capping system as recited in claim 2, wherein the circular seal extends downwardly beyond the circular outside wall such that the seal rim is lower than the cap rim.

7. The universal capping system as recited in claim 2, wherein the circular seal has at least one vertical open slit forming a seal relief.

8. The universal capping system as recited in claim 7, wherein the circular seal has a hook tip disposed near the seal rim.

9. The universal capping system as recited in claim 7, wherein the circular seal has an arcuate shape on an outside surface of the circular seal in a cross-sectional view.

10. The universal capping system as recited in claim 7, wherein the circular seal has a bottom taper portion disposed on a bottom of an outside surface of the circular seal in a cross-sectional view.

11. The universal capping system as recited in claim 10, wherein circular seal has an upper portion having a first inner diameter, and the cap rim has a second inner diameter, wherein the first inner diameter is smaller than the second inner diameter.

12. The universal capping system as recited in claim 6, wherein the circular seal has at least one vertical open slit forming a seal relief.

13. The universal capping system as recited in claim 12, wherein the circular seal has a vertical height sufficiently long to reach beyond a lowest point of a bottle neck when fully fitted onto the bottle finish.

14. The universal capping system as recited in claim 13, wherein circular seal has a hook tip disposed near the seal rim, and the hook tip reaches beyond the lowest point of a bottle neck when fully fitted onto the bottle finish.

15. The universal capping system as recited in claim 6, wherein the bottle-couple part is a detachable douche nozzle.
